(19) United States
(12) Patent Application Publication
(10) Pub. No.: US 2018/0161517 A1
(43) Pub. Date: Jun. 14, 2018

(54) DRUG DELIVERY DEVICE

(71) Applicant: SANOFI-AVENTIS DEUTSCHLAND GMBH, Frankfurt am Main (DE)

(72) Inventor: Markus Ploch, Frankfurt am Main (DE)

(21) Appl. No.: 15/568,744

(22) PCT Filed: Apr. 20, 2016
(86) PCT No.: PCT/EP2016/058738

§ 371 Date: Oct. 23, 2017

(30) Foreign Application Priority Data
Apr. 24, 2015 (EP) 15305637.9

(51) Int. Cl. A61M 5/32; A61M 5/34

(52) U.S. Cl. CPC A61M 5/3213; A61M 2005/3206; A61M 2205/276; A61M 5/34

(57) ABSTRACT

Disclosed is a drug delivery device having an adapter configured for mounting a needle hub and a flange portion configured for cooperating with an orifice of a cap, the cap is configured to cover the adapter, and a latch mechanism configured for releasably engaging a detent or recess in a surface portion of the cap. In addition, the latch mechanism is configured to turn into an inoperable state when a needle hub is mounted to the adapter.

### CROSS REFERENCE TO RELATED APPLICATIONS

The present application is the national stage entry of International Patent Application No. PCT/EP2016/058738, filed on Apr. 20, 2016, and claims priority to Application No. EP 15305637.9, filed in on Apr. 24, 2015, the disclosures of which are expressly incorporated herein in entirety by reference thereto.

### TECHNICAL FIELD

The disclosure relates to a drug delivery device having a latch mechanism configured for releasably engaging a detent or recess in a surface portion of a cap.

### BACKGROUND

In injection devices, there is a trend toward the use of smaller and smaller gauge needles typically used for self-injection. While a small gauge needle typically reduces the pain associated with the procedure, their use can result in an increased risk of partial or complete occlusion of the needle, especially when used in conjunction with higher concentration insulins.

Multi-use drug delivery devices such as insulin pens are typically used with replaceable double-tipped pen needles that are attached to a drug cartridge prior to use. It is generally recommended that the user removes the needle after performing an injection and attaches a new one for the next injection. Otherwise, there is a risk of the user injuring themselves with blunt, bent or clogged needles or delivering an underdose.

One potential cause of needle occlusion is solidification of the drug formulation within the inner bore of the needle. This may occur if the needle is left in-situ following use, or if a user fits a needle in preparation for taking an injection later that day. It is known that particular drug formulations, especially those that are water-based, may be more at risk of solidification in particular storage situations. One such example might be higher concentration insulin formulations.

It may be therefore understood as a requirement with crucial impact to the overall reliability of self-injection to attach an unused needle immediately before injection. There remains a need to help users to easily comply with the aforementioned requirement.

### SUMMARY

In view of the aforementioned need, certain aspects of the present disclosure provide an improved drug delivery device.

Exemplary embodiments of the disclosure are given in the dependent claims.

The term “drug delivery device” shall be understood to encompass any type of device, system or apparatus which may serve to dispense a drug or a formulation containing a drug immediately to a human or veterinarian body. Immediately dispense thereby shall be understood as an absence of any necessary intermediate manipulation of the drug or drug formulation by a user between discharge of the drug or drug formulation from the drug delivery device and administration to the human or veterinarian body. Without limitation, typical examples of an immediate dispensing device may be found in injection devices, inhalers and stomach tube feeding systems.

According to the disclosure, a drug delivery device has an adapter configured for mounting a needle hub and a flange portion configured for cooperating with an orifice of a cap, the cap being configured to cover the adapter, and a latch mechanism configured for releasably engaging a detent or recess in a surface portion of the cap, wherein the latch mechanism is configured to turn into an inoperable state when a needle hub is mounted to the adapter. The cap is thus prevented from being attached or held on the drug delivery device thus indicating to a user that a needle assembly having a needle hub is still attached to the drug delivery device encouraging them to remove the needle assembly before attempting to attach the cap again. This reduces the risk of needle occlusion.

In an exemplary embodiment, the drug delivery device comprises a cartridge holder, e.g. for holding a medicament cartridge, wherein the latch mechanism comprises at least one flexible arm on the cartridge holder, the flexible arm having a protrusion arranged to latch into a respective recess of the cap, the flexible arm arranged to be deflected from a relaxed position into a first deflected position by attaching a needle hub to a distal end of the cartridge holder, wherein in the first deflected position the protrusion is prevented from latching into the recess. The cap is thus prevented from being attached or held on the cartridge holder thus indicating to a user that the needle assembly is still attached to the cartridge holder encouraging them to remove the needle assembly before attempting to attach the cap again. This reduces the risk of needle occlusion.

In other exemplary embodiments the drug delivery device may comprise a medicament cartridge and the latch mechanism, e.g. the flexible arm, may be arranged on the medicament cartridge.

In an exemplary embodiment, in the first deflected position the protrusion is adapted to axially abut the cap preventing the cap from being attached over the distal end of the cartridge holder.

In an exemplary embodiment, in the relaxed state of the arm the protrusion does not axially abut the cap during an attempt to attach it to the cartridge holder thus allowing to assemble the cap over the cartridge holder when no needle assembly is attached.

In an exemplary embodiment, the cartridge holder comprises a body and an adapter distally from the body, the adapter arranged to engage a needle hub of a needle assembly, wherein the flexible arm protrudes from the body in a distal direction in a manner laterally overlapping the adapter.

In an exemplary embodiment, the flexible arm is arranged to be deflected radially inwards from the relaxed position into a second deflected position. This allows for a detent feature in the cap to deflect the arm during assembly of the cap to the cartridge holder prior to the protrusion reaching the recess such that subsequently a detent force has to be overcome to remove the cap so that the cap cannot just fall off if pointed downwards.

In an exemplary embodiment, a ramp feature on a radially inward pointing side of the protrusion is arranged to be engaged by a needle hub of a needle assembly when the needle hub is attached onto the adapter thereby deflecting the arms radially outwards into the first deflected position.

In another exemplary embodiment, in the first deflected position the protrusion allows for attaching the cap over the distal end of the cartridge holder but is prevented from latching into the recess. The protrusion does therefore not engage the recess such that the cap is only loosely arranged over the cartridge holder and does not snap in such that it may fall of if pointed downwards. This indicates that a needle assembly is still attached and encourages a user to remove the needle assembly in order to be able to snap the cap onto the cartridge holder.

In an exemplary embodiment, the arm is prevented from returning from the first deflected position into the relaxed position by a needle assembly attached to the cartridge holder.

In an exemplary embodiment, the cartridge holder comprises a body and an adapter distally from the body, the adapter arranged to engage a needle hub of a needle assembly, wherein the flexible arm is arranged in a cutout within the adapter and protrudes in a proximal direction in a manner laterally overlapping the body.

In an exemplary embodiment, a ramp feature on an radially outward directed surface of the arm is arranged to be engaged by a needle hub of a needle assembly when the needle hub is attached onto the adapter thereby deflecting the arms radially inwards into the first deflected position.

In an exemplary embodiment, one or more longitudinal notches for accommodating the arms are arranged in the cartridge holder thereby defining the possible extent of inward deflection of the arms.

In an exemplary embodiment, the adapter comprises an external thread for engaging an internal thread of a needle hub of a needle assembly.

In another exemplary embodiment, the adapter comprises a bayonet feature for engaging a corresponding bayonet feature of a needle hub of a needle assembly. Likewise, other ways of attaching a needle assembly to the cartridge holder are possible, e.g. friction fit or Luer Lock.

Further scope of applicability of the present disclosure will become apparent from the detailed description given hereinafter. However, it should be understood that the detailed description and specific examples, while indicating exemplary embodiments of the disclosure, are given by way of illustration only, since various changes and modifications within the spirit and scope of the disclosure will become apparent to those skilled in the art from this detailed description.

### BRIEF DESCRIPTION OF THE FIGURES

The present disclosure will become more fully understood from the detailed description given hereinbelow and the accompanying drawings which are given by way of illustration only, and thus, are not limitive of the present disclosure, and wherein:

FIG. 1 is a schematic perspective view of a latch mechanism comprising a cartridge holder,

FIG. 2 is a schematic longitudinal section of a cap assembled over the cartridge holder,

FIG. 3 is a schematic perspective view of the cartridge holder with an attached needle assembly,

FIG. 4 is a schematic longitudinal section of the cartridge holder with the attached needle assembly during an attempt to assemble a cap over the cartridge holder,

FIG. 5 is a schematic perspective view of another embodiment of a latch mechanism comprising a cartridge holder,

FIG. 6 is a schematic longitudinal section of a cap assembled over the cartridge holder,

FIG. 7 is a schematic perspective view of the cartridge holder with an attached needle assembly,

FIG. 8 is a schematic longitudinal section of the cartridge holder with the attached needle assembly during an attempt to assemble a cap over the cartridge holder, and

FIG. 9 is a schematic view of a drug delivery device with a needle assembly and a cap.

Corresponding parts are marked with the same reference symbols in all figures.

### DETAILED DESCRIPTION

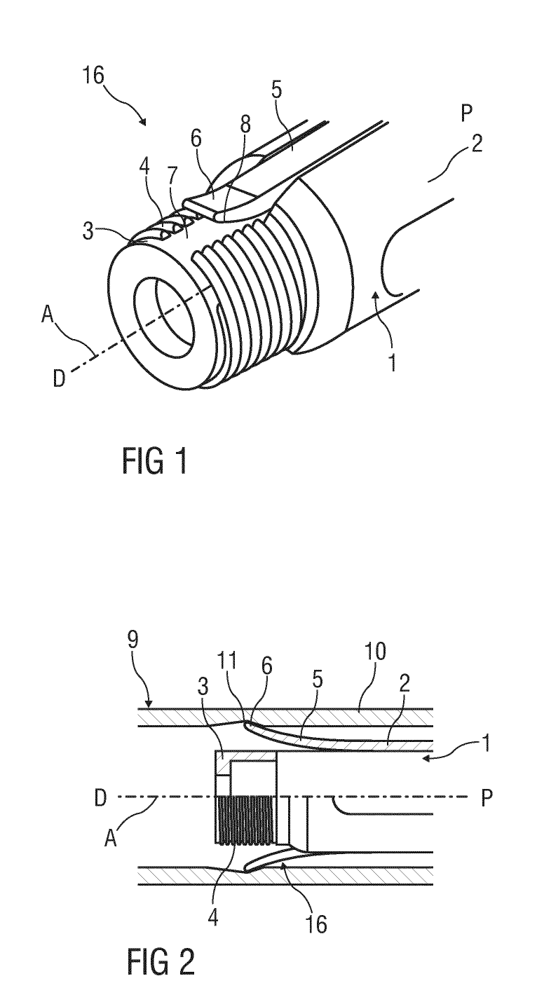

FIG. 1 is a schematic perspective view of a latch mechanism 16 comprising a cartridge holder 1. The cartridge holder 1 comprises a body 2 adapted to receive a medicament cartridge (not illustrated) arranged for containing a fluid to be injected. A distal end of the cartridge may comprise a septum. Distally from the body 2, the cartridge holder 1 comprises an adapter 3 with an external thread 4 for engaging an internal thread of a needle hub of a needle assembly. One or more flexible arms 5 protrude from the body 2 in a distal direction D in a manner laterally overlapping the adapter 3. At their distal ends, the flexible arms 5 comprise a protrusion 6 protruding radially outwards, i.e. away from a longitudinal axis A of the cartridge holder 1. In a relaxed position as shown in FIG. 1, the protrusions 6 of the arms 5 define a diameter that is sufficiently small to allow attaching a cap over the distal end of the cartridge holder 1. The flexibility of the arms 5 allows for deflecting them radially outwards into a first deflected position thereby increasing the diameter defined by the protrusions 6 and radially inwards into a second deflected position thereby reducing the diameter defined by the protrusions 6. In an exemplary embodiment, one or more longitudinal notches 7 for accommodating the arms 5 are arranged in the adapter 3 thereby defining the possible extent of inward deflection of the arms 5 in the second deflected position. A ramp feature 8 on a radially inward pointing side of the protrusion 6 is arranged to be engaged by a needle hub of a needle assembly when the needle hub is screwed onto the adapter 3 thereby deflecting the arms 5 radially outwards into the first deflected position.

In FIG. 1, a needle assembly is not attached to the adapter 3, the arms 5 are hence in their relaxed position such that a cap can be attached over the cartridge holder 1.

FIG. 2 is a schematic longitudinal section of a cap 9 assembled over the cartridge holder 1. During assembly the protrusions 6 slide along an inner surface of a wall 10 of the cap 9 until arriving at a recess 11 in the wall 10 allowing for engagement of the protrusions 6 into the recess 11 with the arms 5 in the relaxed position or only slightly deflected towards the second deflected position. In an exemplary embodiment, before arriving at the recess 11, the protrusions have been radially inwardly deflected towards the second deflected position due to a respective design of the wall 10. For example, the wall 10 may be designed to decrease an internal diameter of the cap 9 in the distal direction D until reaching the recess 11. Likewise, the wall 10 may be designed to define a substantially constant diameter and a bump feature, i.e. a local reduction in diameter could be arranged adjacent the recess 11 in a proximal direction P. Likewise, a funnel feature could be arranged at a proximal end of the cap 9 for radially inwardly deflecting the protrusions 6 upon assembly of the cap 9 over the cartridge holder 1 and the wall 10 may be designed to define a substantially constant diameter between the funnel feature and the recess 11. The engagement of the protrusions 6 in the recesses 11 keeps the cap 9 attached on the cartridge holder 1 such that a detent force has to be overcome to remove the cap 9.

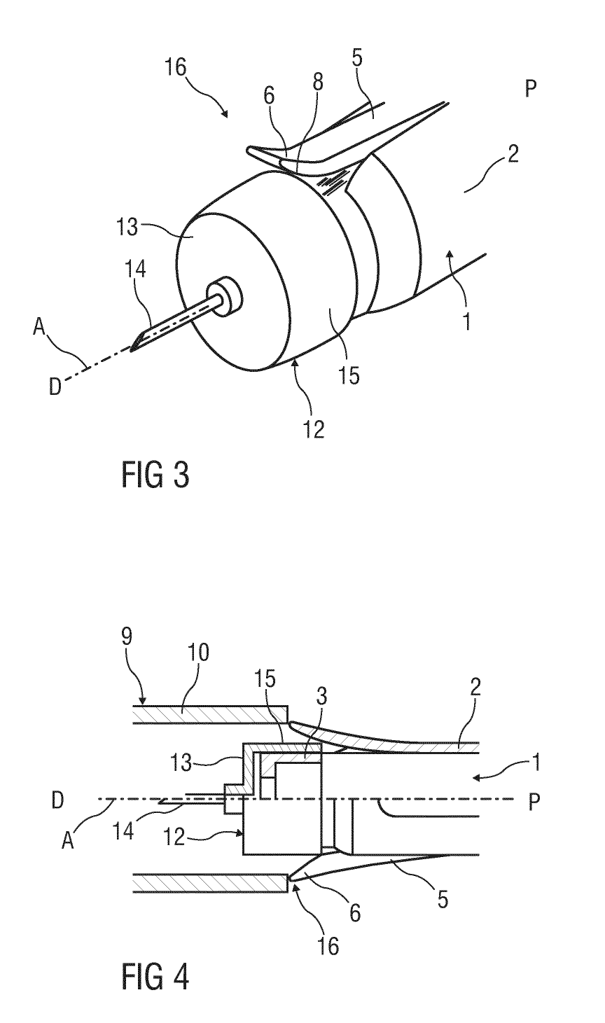

FIG. 3 is a schematic perspective view of the cartridge holder 1 with an attached needle assembly 12. The needle assembly 12 comprises a needle hub 13 and a hypodermic double ended hollow needle 14 held in the needle hub 13. The needle hub 13 comprises a cylindrical wall 15 with an internal thread (not illustrated) adapted to engage the external thread 4 of the adapter 3. As the needle hub 13 is screwed onto the adapter 3, a proximal tip of the needle 14 may pierce a septum of a cartridge held within the cartridge holder 1 establishing a fluid communication between the cartridge and the needle 14. Further, as the needle hub 13 is screwed onto the adapter 3 the wall 15 engages the ramp features 8 of the flexible arms 5 displacing the arms 5 radially outwards into the first deflected position thereby increasing the diameter defined by the protrusions 6.

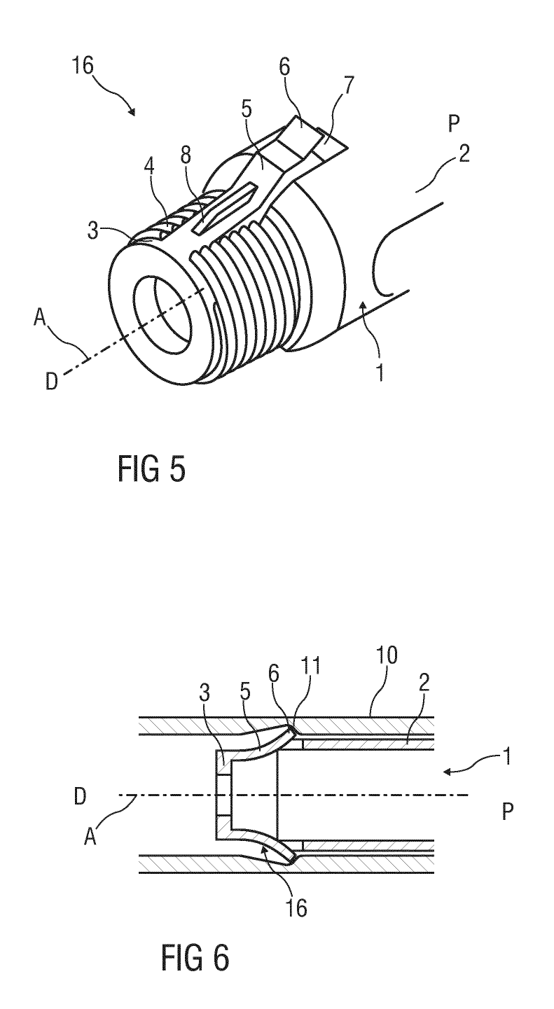

FIG. 4 is a schematic longitudinal section of the cartridge holder 1 with the attached needle assembly 12 during an attempt to assemble a cap 9 over the cartridge holder 1. The diameter defined by the protrusions 6 with the arms in the first deflected position is greater than an internal diameter of the wall 10 of the cap 9 at a proximal end thereof. The cap 9 can thus not be assembled over the cartridge holder 1. This indicates that a needle assembly 12 is still attached and encourages a user to remove the needle assembly 12 in order to be able to attach the cap 9.

FIG. 5 is a schematic perspective view of another embodiment of a latch mechanism 16 comprising a cartridge holder 1. The cartridge holder 1 comprises a body 2 adapted to receive a medicament cartridge (not illustrated) arranged for containing a fluid to be injected. A distal end of the cartridge may comprise a septum. Distally from the body 2, the cartridge holder 1 comprises an adapter 3 with an external thread 4 for engaging an internal thread of a needle hub of a needle assembly. One or more flexible arms 5 are arranged in a cutout within the adapter 3 and protrude in a proximal direction P in a manner laterally overlapping the body 2. At their proximal ends, the flexible arms 5 comprise respective protrusions 6 protruding radially outwards, i.e. away from a longitudinal axis A of the cartridge holder 1. In FIG. 5, the arms 5 are in a relaxed position. The flexibility of the arms 5 allows for deflecting them radially inwards into a first deflected position thereby reducing the diameter defined by the protrusions 6. In an exemplary embodiment one or more longitudinal notches 7 or recesses for accommodating the arms 5 are arranged in the body 2 thereby defining the possible extent of inward deflection of the arms 5. A ramp feature 8 on an external surface of the arm 5 in the area of the adapter 3 is arranged to be engaged by a needle hub of a needle assembly when the needle hub is screwed onto the adapter 3 thereby deflecting the arms 5 radially inwards into the first deflected position.

In FIG. 5, a needle assembly is not attached to the adapter 3, the arms 5 are hence in their relaxed position such that a cap can be attached over the cartridge holder 1.

FIG. 6 is a schematic longitudinal section of a cap 9 assembled over the cartridge holder 1. During assembly of the cap 9 the protrusions 6 slide along an inner surface of a wall 10 of the cap 9 until arriving at a recess 11 in the wall 10 allowing for engagement of the protrusions 6 into the recess 11 with the arms 5 in the relaxed position. In an exemplary embodiment, before arriving at the recess 11, the protrusions have been radially inwardly deflected towards the first deflected position due to a respective design of the wall 10. For example, the wall 10 may be designed to decrease an internal diameter of the cap 9 in the distal direction D until reaching the recess 11. Likewise, the wall 10 may be designed to define a substantially constant diameter and a bump feature, i.e. a local reduction in diameter could be arranged adjacent the recess 11 in a proximal direction P. Likewise, a funnel feature could be arranged at a proximal end of the cap 9 for radially inwardly deflecting the protrusions 6 upon assembly of the cap 9 over the cartridge holder 1 and the wall 10 may be designed to define a substantially constant diameter between the funnel feature and the recess 11. The engagement of the protrusions 6 in the recesses 11 keeps the cap 9 attached on the cartridge holder 1 such that a detent force has to be overcome to remove the cap 9.

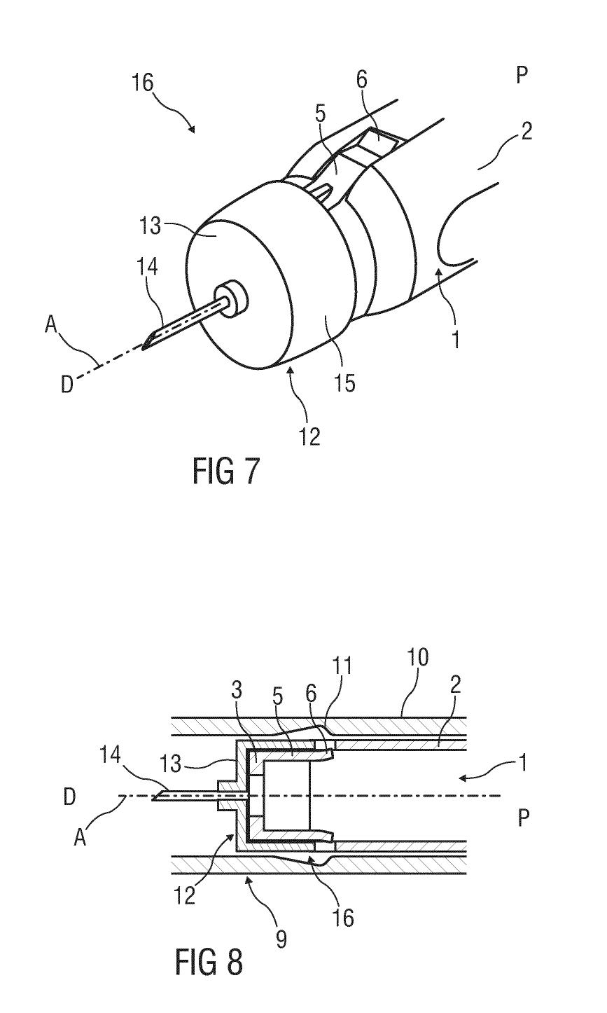

FIG. 7 is a schematic perspective view of the cartridge holder 1 with an attached needle assembly 12.

The needle assembly 12 comprises a needle hub 13 and a hypodermic double ended hollow needle 14 held in the needle hub 13. The needle hub 13 comprises a cylindrical wall 15 with an internal thread (not illustrated) adapted to engage the external thread 4 of the adapter 3. As the needle hub 13 is screwed onto the adapter 3 a proximal tip of the needle 14 may pierce a septum of a cartridge held within the cartridge holder 1 establishing a fluid communication between the cartridge and the needle 14. Further, as the needle hub 13 is screwed onto the adapter 3 the wall 15 engages the ramp features 8 of the flexible arms 5 displacing the arms 5 radially inwards into the first deflected position thereby decreasing the diameter defined by the protrusions 6.

FIG. 8 is a schematic longitudinal section of the cartridge holder 1 with the attached needle assembly 12 during an attempt to assemble a cap 9 over the cartridge holder 1. During an attempt to assemble the cap 9 over the cartridge holder 1, the diameter defined by the protrusions 6 with the arms 5 in the first deflected position is smaller than required for engaging the recesses 11 in the wall 10 of the cap 9. The protrusions 6 do therefore not engage the recesses 11 such that the cap 9 is only loosely arranged over the cartridge holder 1 and does not snap in such that it may fall of if pointed downwards. This indicates that a needle assembly 12 is still attached and encourages a user to remove the needle assembly 12 in order to be able to snap the cap 9 onto the cartridge holder 1.

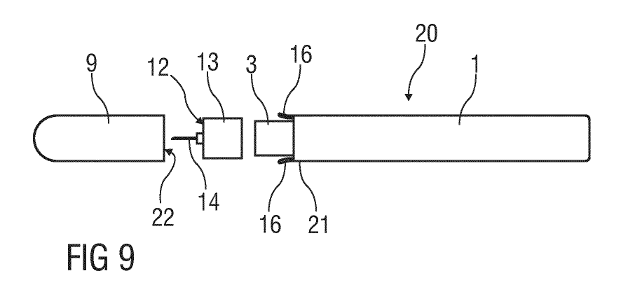

FIG. 9 is a schematic view of a drug delivery device 20 with a needle assembly 12 and a cap 9. 1. The drug delivery device 20 has an adapter 3 configured for mounting a needle hub 13 of the needle assembly 12 and a flange portion 21 configured for cooperating with an orifice 22 of the cap 9, the cap 9 being configured to cover the adapter 3, and a latch mechanism 16 configured for releasably engaging a detent or recess (cf. FIGS. 2, 6, 8) in a surface portion of the cap 9, wherein the latch mechanism 16 is configured to turn into an inoperable state when a needle hub 13 is mounted to the adapter 3. The drug delivery device 20 may comprise a cartridge holder 1, on which the adapter 3 and the latch mechanism 16 are provided. In other embodiments the drug delivery device 20 may comprise a medicament cartridge, on which the adapter 3 and the latch mechanism 16 may be provided.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivatives are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(w-carboxyheptadecanoyl)-des(B30) human insulin and B29-N-(w-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser- Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2, H-(Lys)5-des Pro36, des Pro37 Exendin-4(1-39)-NH2, des Pro36 Exendin-4(1-39), des Pro36 [Asp28] Exendin-4(1-39), des Pro36 [IsoAsp28] Exendin-4(1-39), des Pro36 [Met(O)14, Asp28] Exendin-4(1-39), des Pro36 [Met(O)14, IsoAsp28] Exendin-4(1-39), des Pro36 [Trp(O2)25, Asp28] Exendin-4(1-39), des Pro36 [Trp(O2)25, IsoAsp28] Exendin-4(1-39), des Pro36 [Met(O)14 Trp(O2)25, Asp28] Exendin-4(1-39), des Pro36 [Met(O)14 Trp(O2)25, IsoAsp28] Exendin-4(1-39); or

des Pro36 [Asp28] Exendin-4(1-39), des Pro36 [IsoAsp28] Exendin-4(1-39), des Pro36 [Met(O)14, Asp28] Exendin-4(1-39), des Pro36 [Met(O)14, IsoAsp28] Exendin-4(1-39), des Pro36 [Trp(O2)25, Asp28] Exendin-4(1-39), des Pro36 [Trp(O2)25, IsoAsp28] Exendin-4(1-39), des Pro36 [Met(O)14 Trp(O2)25, Asp28] Exendin-4(1-39), des Pro36 [Met(O)14 Trp(O2)25, IsoAsp28] Exendin-4(1-39), wherein the group-Lys6-NH2 may be bound to the C-terminus of the Exendin-4 derivative;

or an Exendin-4 derivative of the sequence des Pro36 Exendin-4(1-39)-Lys6-NH2 (AVE0010), H-(Lys)6-des Pro36 [Asp28] Exendin-4(1-39)-Lys6-NH2, des Asp28 Pro36, Pro37, Pro38Exendin-4(1-39)-NH2, H-(Lys)6-des Pro36, Pro38 [Asp28] Exendin-4(1-39)-NH2, H-Asn-(Glu)5des Pro36, Pro37, Pro38 [Asp28] Exendin-4(1-39)-NH2, des Pro36, Pro37, Pro38 [Asp28] Exendin-4(1-39)-(Lys)6-NH2, H-(Lys)6-des Pro36, Pro37, Pro38 [Asp28] Exendin-4(1-39)-(Lys)6-NH2, H-Asn-(Glu)5-des Pro36, Pro37, Pro38 [Asp28] Exendin-4(1-39)-(Lys)6-NH2, H-(Lys)6-des Pro36 [Trp(O2)25, Asp28] Exendin-4(1-39)-Lys6-NH2, H-des Asp28 Pro36, Pro37, Pro38 [Trp(O2)25] Exendin-4(1-39)-NH2, H-(Lys)6-des Pro36, Pro37, Pro38 [Trp(O2)25, Asp28] Exendin-4(1-39)-NH2, H-Asn-(Glu)5-des Pro36, Pro37, Pro38 [Trp(O2)25, Asp28] Exendin-4(1-39)-NH2, des Pro36, Pro37, Pro38 [Trp(O2)25, Asp28] Exendin-4(1-39)-(Lys)6-NH2, H-(Lys)6-des Pro36, Pro37, Pro38 [Trp(O2)25, Asp28] Exendin-4(1-39)-(Lys)6-NH2, H-Asn-(Glu)5-des Pro36, Pro37, Pro38 [Trp(O2)25, Asp28] Exendin-4(1-39)-(Lys)6-NH2, H-(Lys)6-des Pro36 [Met(O)14, Asp28] Exendin-4(1-39)-Lys6-NH2, des Met(O)14 Asp28 Pro36, Pro37, Pro38 Exendin-4(1-39)-NH2, H-(Lys)6-desPro36, Pro37, Pro38 [Met(O)14, Asp28] Exendin-4(1-39)-NH2, H-Asn-(Glu)5-des Pro36, Pro37, Pro38 [Met(O)14, Asp28] Exendin-4(1-39)-NH2, des Pro36, Pro37, Pro38 [Met(O)14, Asp28] Exendin-4(1-39)-(Lys)6-NH2, H-(Lys)6-des Pro36, Pro37, Pro38 [Met(O)14, Asp28] Exendin-4(1-39)-(Lys)6-NH2, H-Asn-(Glu)5 des Pro36, Pro37, Pro38 [Met(O)14, Asp28] Exendin-4(1-39)-(Lys)6-NH2, H-Lys6-des Pro36 [Met(O)14, Trp(O2)25, Asp28] Exendin-4(1-39)-Lys6-NH2, H-des Asp28 Pro36, Pro37, Pro38 [Met(O)14, Trp(O2)25] Exendin-4(1-39)-NH2, H-(Lys)6-des Pro36, Pro37, Pro38 [Met(O)14, Asp28] Exendin-4(1-39)-NH2, H-Asn-(Glu)5-des Pro36, Pro37, Pro38 [Met(O)14, Trp(O2)25, Asp28] Exendin-4(1-39)-NH2, des Pro36, Pro37, Pro38 [Met(O)14, Trp(O2)25, Asp28] Exendin-4(1-39)-(Lys)6-NH2, H-(Lys)6-des Pro36, Pro37, Pro38 [Met(O)14, Trp(O2)25, Asp28] Exendin-4(S1-39)-(Lys)6-NH2, H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, σ, ε, γ, and μ. The type of heavy chain present defines the isotype of antibody; these chains are found in IgA, IgD, IgE, IgG, and IgM antibodies, respectively.

Distinct heavy chains differ in size and composition; a and y contain approximately 450 amino acids and σ approximately 500 amino acids, while p and E have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, a and σ have a constant region composed of three tandem Ig domains, and a hinge region for added flexibility; heavy chains p and E have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by A and K. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCI or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

Those of skill in the art will understand that modifications (additions and/or removals) of various components of the apparatuses, methods and/or systems and embodiments described herein may be made without departing from the full scope and spirit of the present disclosure, which encompass such modifications and any and all equivalents thereof.

### LIST OF REFERENCES

1 cartridge holder

2 body

3 adapter

4 external thread

5 flexible arm

6 protrusion

7 notch

8 ramp feature

9 cap

10 wall

11 recess

12 needle assembly

13 hub

14 needle

15 wall

16 latch mechanism

20 drug delivery device

21 flange portion

22 orifice

A longitudinal axis

D distal direction

P proximal direction

1-14. (canceled)

15. A drug delivery device comprising:
an adapter configured for mounting a needle hub;
a flange portion configured for cooperating with an orifice of a cap, the cap being configured to cover the adapter; and
a latch mechanism configured for releasably engaging a detent or recess in a surface portion of the cap,
wherein the latch mechanism is configured to change into an inoperable state when a needle hub is mounted to the adapter.

16. The drug delivery device according to claim 15, comprising a cartridge holder, wherein the latch mechanism comprises a flexible arm on the cartridge holder, the flexible arm having a protrusion arranged to latch into a respective recess of the cap, the flexible arm arranged to be deflected from a relaxed position into a first deflected position by attaching a needle hub to a distal end of the cartridge holder, wherein in the first deflected position the protrusion is prevented from latching into the recess.

17. The drug delivery device according to claim 16, wherein, in the first deflected position, the protrusion is adapted to axially abut the cap preventing the cap from being attached over the distal end of the cartridge holder.

18. The drug delivery device according to claim 16, wherein in the relaxed state the protrusion does not axially abut the cap during an attempt to attach it to the cartridge holder.

19. The drug delivery device according to claim 16, wherein the cartridge holder comprises a body and an adapter distally from the body, the adapter arranged to engage a needle hub of a needle assembly, wherein the flexible arm protrudes from the body in a distal direction in a manner laterally overlapping the adapter.

20. The drug delivery device according to claim 16, wherein the flexible arm is arranged to be deflected radially inwards from the relaxed position into a second deflected position.

21. The drug delivery device according to claim 16, wherein a ramp feature on a radially inward pointing side of the protrusion is arranged to be engaged by a needle hub of a needle assembly when the needle hub is attached onto the adapter thereby deflecting the flexible arm radially outwards into the first deflected position.

22. The drug delivery device according to claim 16, wherein in the first deflected position the protrusion allows for attaching the cap over the distal end of the cartridge holder but is prevented from latching into the recess.

23. The drug delivery device according to claim 16, wherein the flexible arm is prevented from returning from the first deflected position into the relaxed position by a needle assembly attached to the cartridge holder.

24. The drug delivery device according to claim 16, wherein the cartridge holder comprises a body and an adapter distally from the body, the adapter arranged to engage a needle hub of a needle assembly, wherein the flexible arm is arranged in a cutout within the adapter and protrudes in a proximal direction in a manner laterally overlapping the body.

25. The drug delivery device according to claim 16, wherein a ramp feature on an radially outward directed surface of the flexible arm is arranged to be engaged by a needle hub of a needle assembly when the needle hub is attached onto the adapter thereby deflecting the flexible arm radially inwards into the first deflected position.

26. The drug delivery device according to claim 16, wherein one or more longitudinal notches configured to accommodate the flexible arm are arranged in the cartridge holder.

27. The drug delivery device according to claim 19, wherein the adapter comprises an external thread configured to engage an internal thread of a needle hub of a needle assembly.

28. The drug delivery device according to claim 19, wherein the adapter comprises a bayonet feature configured to engage a corresponding bayonet feature of a needle hub of a needle assembly.

29. The drug delivery device according to claim 15, comprising a cartridge carrying a medicament having a pharmaceutically active compound.
